(19) United States
(12) Patent Application Publication
(10) Pub. No.: US 2025/0161559 A1
(43) Pub. Date: May 22, 2025

(54) MEMS MICROPUMP WITH PIEZOELECTRIC VALVE IN UNACTUATED STATE THAT REMAINS CLOSED FOLLOWING MICROPUMP POWER LOSS

(71) Applicant: AITA BIO INC., New York, NY (US)

(72) Inventors: Laurence Heads, Frisco, TX (US); Jacob Menture, New York, NY (US)

(21) Appl. No.: 18/834,018

(22) PCT Filed: Feb. 13, 2023
(86) PCT No.: PCT/US2023/012946

§ 371 Date: Jul. 29, 2024

Related U.S. Application Data
(60) Provisional application No. 63/311,445, filed on Feb. 17, 2022.

(51) Int. Cl. A61M 5/142; F04B 43/04; F04B 43/073

(52) U.S. Cl. CPC A61M 5/14224; F04B 43/046; F04B 43/073; A61M 5/14248; A61M 2205/0244; A61M 2205/0294; A61M 2230/201

(57) ABSTRACT

A MEMS device for a device for delivering medicament to a user, the MEMS device comprising: a first port and a second port to enable medicament to flow through the MEMS device; first and second wafers that define a cavity therebetween that communicates with the first and second ports, thereby creating a fluid path for a flow of the medicament from the first port to the second port, the first wafer configured as a membrane adapted to deform, the cavity including a first chamber that communicates with the second port; a first valve section including a first valve seat around the first port; and a first piezoelectric actuator layered on the first wafer and configured to cause the membrane to deform and close off the first valve seat, thereby preventing fluid flow through the first port.

### CROSS REFERENCE TO RELATED APPLICATIONS

This application claims priority to U.S. provisional application No. 63/311,445, filed on Feb. 17, 2022 entitled “Mems Micropump With Piezoelectric Valve In Unactuated State That Remains Closed Following Micropump Power Loss” which is incorporated by reference herein.

### FILED OF THE INVENTION

The present invention relates to a MEMS micropump with piezoelectric valve in unactuated state that remains closed following micropump power loss.

### BACKGROUND OF THE INVENTION

Insulin delivery devices help people with diabetes to conveniently manage their blood sugar. These devices deliver insulin at specific times. Insulin patch pumps or pods are one type of insulin pump. The pods are wearable devices that adhere to the skin of a user using an adhesive patch. The pods incorporate a pump for delivering insulin from through a chamber and internal cannula based on separately acquired CGM sensor readings. It is important to make sure that these pods deliver the correct amount of insulin times to avoid serious harm to the user. It would be advantageous to provide improvements to these insulin pumps to ensure that only the correct amount of insulin is delivered.

### SUMMARY OF THE INVENTION

The MEMS micropump with piezoelectric valve in unactuated state that remains closed following micropump power loss is disclosed.

In accordance with an embodiment of the present disclosure, a MEMS device for a device for delivering medicament to a user, the MEMS device configured as a valve for permitting or preventing a flow of the medicament to the user, the MEMS device comprising: a first port and a second port to enable medicament to flow through the MEMS device; first and second wafers that define a cavity therebetween that communicates with the first and second ports, thereby creating a fluid path for a flow of the medicament from the first port to the second port, the first wafer configured as a membrane adapted to deform, the cavity including a first chamber that communicates with the second port; a first valve section including (1) a first valve seat around the first port that extends from the second wafer into the first chamber to a distal end thereof and (2) a valve gap that is defined as a distance between the distal end and the membrane, wherein the valve seat is configured so that a hydraulic resistance through the valve gap exceeds a hydraulic resistance in the valve section to ensure that medicament is prevented from flowing through the first port in the event the MEMS device has lost power; and a first piezoelectric actuator layered on the first wafer and configured to cause the membrane to deform and close off the first valve seat, thereby preventing fluid flow through the first port.

In accordance with another embodiment of the disclosure, a device for delivering medicament to a user including a MEMS device configured as a micropump for pumping the medicament into the user, the MEMS device comprising: a first port and a second port to enable medicament to flow through the MEMS device; first and second wafers that define a cavity therebetween that communicates with the first and second ports, thereby creating a fluid path for a flow of the medicament through the first port and the second port, the first wafer configured as a membrane that that is adapted to deform into the cavity, the cavity including a first chamber that communicates with the first port and a second chamber that communicates with the first chamber and the second port creating the fluid path and enabling the flow of medicament through the MEMS device; a pump section including a first piezoelectric actuator that is layered on top of the first wafer and is configured to deform the first wafer into the first chamber to draw into or displace medicament into the first chamber; and a first valve section including a first valve seat around the second port that extends from the second wafer into the second chamber to a distal end thereof and (2) a valve gap that is defined as a distance between the distal end and the membrane, wherein the valve seat is configured so that a hydraulic resistance through the valve gap exceeds a hydraulic resistance in the valve section to ensure that medicament is prevented from flowing through the second port in the event the MEMS device has lost power, wherein the second valve section further includes a second piezoelectric actuator layered on the first wafer and configured to cause the membrane to deform and close off the first valve seat, thereby preventing fluid flow through the second port.

In accordance with another embodiment of the disclosure, a method of actuating a MEMS device for a device for delivering medicament to a user, the MEMS device configured as a valve for permitting or preventing a flow of the medicament to the user, the MEMS device including a first port and a second port to enable medicament to flow through the MEMS device, first and second wafers that define a cavity therebetween that communicates with the first and second ports, thereby creating a fluid path for a flow of the medicament from the first port to the second port, the first wafer configured as a membrane, the cavity including a first chamber that communicates with the second port, the MEMS device further including (1) a first valve section with a first valve seat around the second port that extends from the second wafer into the second chamber to a distal end thereof, (2) a valve gap that is defined as a distance between the distal end and the membrane and (3) a first piezoelectric actuator layered on the first wafer to cause the membrane to deform and seal the second port, thereby preventing fluid flow through the second port, the method comprising: adjusting the dimensions of the valve seat so that a hydraulic resistance through the valve gap exceeds a hydraulic resistance in the MEMS device to ensure that medicament is prevented from flowing through the second port in the event the MEMS device has lost power.

### BRIEF DESCRIPTION OF DRAWINGS

FIG. 1 depicts a perspective exploded view of an example micropump or pump 100.

FIG. 2 depicts a cross sectional view of the micropump in FIG. 1 in fully formed configuration.

FIG. 3 depicts an enlarged sectional view of a valve section shown in FIG. 2 along line 3-3.

FIG. 4 depicts a block diagram of example components of device for delivering insulin.

### DETAILED DESCRIPTION OF THE INVENTION

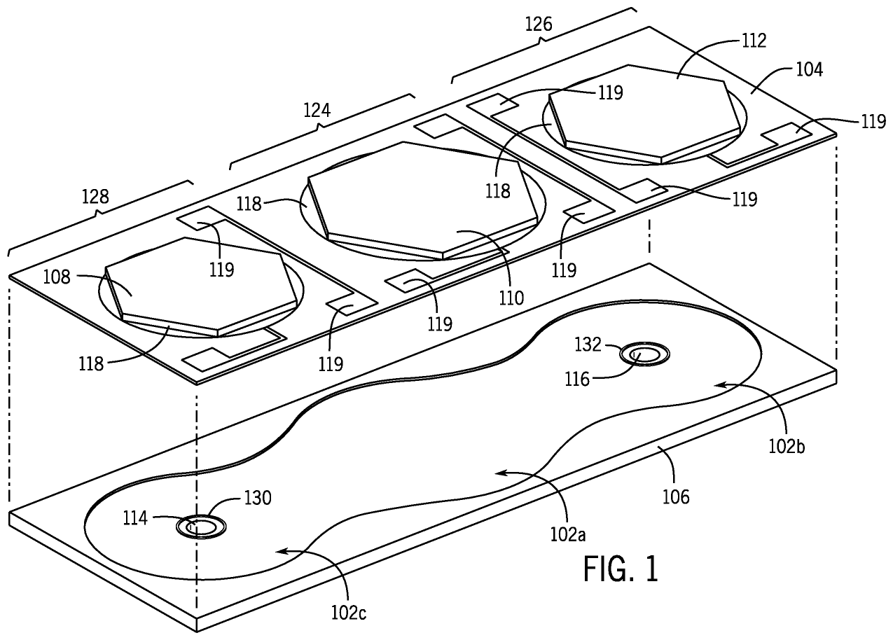

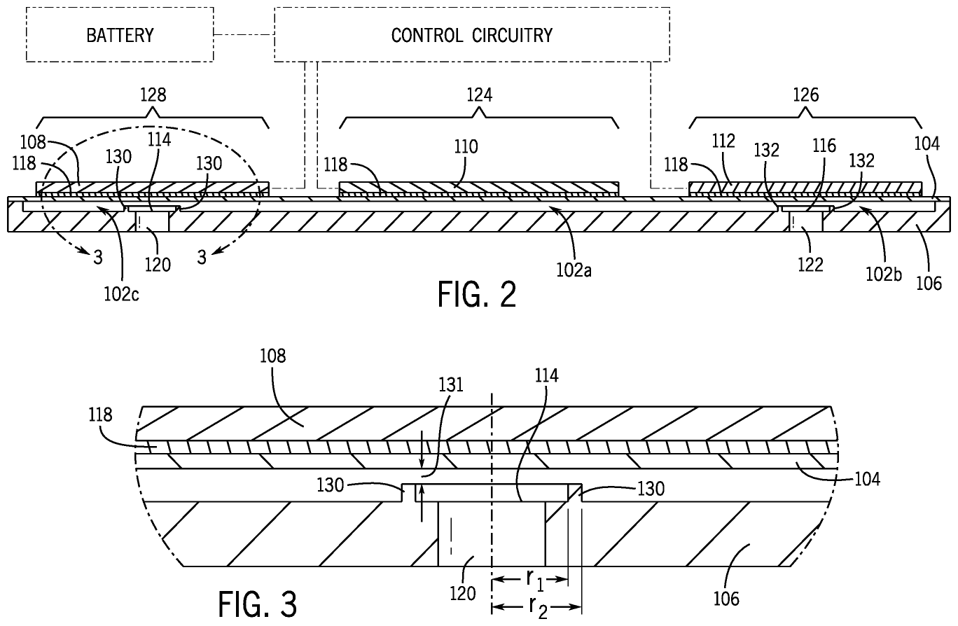

FIG. 1 depicts a perspective exploded view of an example micropump or pump 100. Micropump 100 is part (component) of a device for delivering insulin or other fluid medicament (device 400 described below). The device that is configured as a wearable apparatus or system, of an infusion system for diabetes management, in which continuous glucose monitoring (CGM), insulin delivery and control functionality are provided to ensure insulin is delivered at very precise rates. Device 100 includes several components or modules (not shown) including, among other components, a reservoir for storing the insulin, control circuitry (integrated circuit—IC), battery for powering the IC, an insulin needle and a continuous glucose monitoring (CGM) sensor.

Micropump 100 is a MEMS (micro-electro-mechanical systems) device, as known to those skilled in the art, that can be used for pumping fluid, valves used for regulating flow, actuators used for moving or controlling the micropump and valves and/or sensors used for sensing pressure and/or flow. The MEMS device incorporates one or more piezoelectric elements or devices (also known herein as piezoelectric transducers), as known to those skilled in the art. Example piezoelectric devices include piezoelectric actuators and various types of MEMS sensors. As described in more detail below, the piezoelectric devices function as the active element(s) of a pump for pumping fluid and valves for preventing fluid flow and/or a sensor for sensing pressure or flow. (However, various types of MEMS sensors can be used as the sensing elements of the architecture.) Further, other MEMS or non-MEMS structures or technology may also be used to achieve desired results as known to those skilled in the art.) Micropump 100 may be used in a drug infusion system for infusing a drug (i.e., medication) or other fluid to a patient (user). Medication may include small molecule pharmaceutical solutions, large molecule or protein drug solutions, saline solutions, blood or other fluids known to those skilled in the art. Insulin is an example fluid and described below with respect to micropump 100. However, micropump 100 may be used in other environments known to those skilled in the art.

Micropump 100 is configured to maximize micropump efficiency per mm² (i.e., stroke volume per unit area per Watt). To this end, micropump 100 is a cavity substrate that includes cavity 102 comprising three chambers 102a, 102b and 102c for fluid flow. Micropump 100 further includes (1) silicon on insulator (SOI) wafer 104 (top wafer) that functions as a membrane for chambers 102a, 102b, 102c. SOI wafer 104 incorporates a buried oxide (BOX) layer and a silicon (Si) layer as known to those skilled in the art and (2) double sided polish (DSP) silicon wafer or layer 106. The handle silicon layer of the SOI wafer is removed to form the pump membrane. SOI wafer 104 sits between silicon wafer 106 and several piezoelectric actuators (transducers) 108, 110, 112 as shown and described below in more detail. A metallization and conductive epoxy layer 118 binds piezoelectric actuators 108, 110 and 112 to SOI wafer 104 as known to those skilled in the art. In some detail, certain portions of layer 118 underneath corresponding piezoelectric actuators 108, 110, 112 act as ground electrodes while bonding pads 119 function as active electrodes as known to those skilled in the art. Wafer 106 includes inlet and outlet ports 114, 116 that communicate with chambers 102b and 102c of cavity 102 via channels 120, 122, respectively, that extend through the combined wafer structure (wafers 104, 106) as shown. (Note that wafers 106 may alternatively be SOI wafers as known to those skilled in the art.).

Micropump 100 includes pump section 124 and two valve sections 126, 128 that function together to pump fluid through cavity chambers 102a, 102b, 102c of micropump 100. Pump section 124 includes piezoelectric actuator 110 that is layered on top of silicon layer 104 (via metallization layer 118) and it functions to pump or deform/bend silicon layer 104 to draw into or displace liquid contents into cavity chamber 102a from either port 114 or port 116 as desired.

Valve sections 126, 128 are configured as piezoelectric valves, i.e., microvalves that function as active valves as described in more detail below. Valve sections 122, 124 include piezoelectric actuators 108, 112 respectively, as well as valve seats 130, 132, respectively. As described in more detail below, valve seats 130, 132 are configured as an annular or circular ring that extends from the second wafer 106 into cavity chambers 102b, 102c and define the introduction of channels 120, 122 and inlet/outlet ports 114, 116. As described above, piezoelectric actuators 108, 112 are layered on top of SOI wafer 104 (via metallization layer 118). Piezoelectric actuators 108, 112 are configured to compress against SOI wafer 104 (membrane) and wafer 104 deforms to reach and seal valve seats 130, 132 to thereby discontinue flow through inlet/outlets 114, 116, respectively as needed for proper pump performance, as known to those skilled in the art. (Note that a micropump may include any number of pumps and/or valves as described herein.)

As described above, valve sections 122, 124 are configured as piezoelectric microvalves that function as active valves. In this respect, these valves require activation to be closed in order to prevent free fluid flow which can cause additional drain in power consumption of the system. This is especially important when considering wearable devices.

In this example, valve sections 122, 124 are part of three chamber micropump 100 as described above and shown, but any number of chambers may be used to achieve desired results. Valve chambers 102b, 102c are defined by a two silicon wafer structure. The main factor that impedes fluid flow through micropump 100 is hydraulic resistance, specifically hydraulic resistance Rh through a pipe or chamber, i.e., chamber hydraulic resistance given by:

$$R h = \frac{12 η l}{w {h}^{3}} \qquad ( 1 )$$

where n is fluid viscosity, l is the length of the “channel” or thickness of the valve seat (i.e., l=r₁−r₂), (h) is valve gap height or distance, w is the width of the “channel” or the circumference of the lip of the valve seat.

Pressure drop Δp_{1→2} through a viscous slit flow profile, which can be assumed when a valve seat width w is greater than the valve gap height h is given by:

$$Δ {p}_{1 \to 2} = \frac{6 η}{π {h}^{3}} ln ( \frac{{r}_{2}}{{r}_{1}} ) q \qquad ( 2 )$$

where q is flow rate.

The main geometry that is adjusted to derive a normally closed valve is the geometry of the valve seat, which is a raised circular or annular ring extending from the bottom of wafer 106 into the chamber as shown (FIG. 1 best illustrates) that surrounds inlet and outlet ports 120, 122. Valve seats 130, 132 each have a valve lip that is the distal end of the valve seat (away from wafer 106). As best shown in the example in FIG. 3, valve gap 131 is the distance between the lip of valve seat 130 and the surface of wafer 104 facing the chamber 102c. By changing the dimensions of this valve seat (given the viscosity of fluid in this example), the hydraulic resistance through the valve gap dominates that of the valve chamber and the rest of micropump 100 by orders of magnitude and is therefore of importance (and for calculations). (The height of the valve seat is preferably between 0.05-10 μm in the unactuated state, however, valve seat height may be outside of the range to achieve desired results as known to those skilled in the art. For example, valve seat height may be 20 μm, 50 μm or 100 μm depending on various factors such as viscosity and other properties of fluid and the surface energy of the micropump.) Another important factor in the geometry is the width of the valve seat l as described above which is represented by l=r₂−r₁ as described above. By varying the valve gap distance, the hydraulic resistance of the valve may be tuned further, i.e., increasing the width would further increase the hydraulic resistance, and vice versa. Finally, the radius of the valve seat may also be adjusted which when increased results as follows: 1) it reduces hydraulic resistance as the area in which the fluid flows is increased but 2) means that the gap height between the valve seat (lip) and the membrane decreases as the deflection or deformation of a circular silicon membrane is parabolic from the center (and vice versa).

By deriving the pressure drop across chosen valve dimensions, the forward pressure that the valve can withstand, without free flow, can be demonstrated. Given that there are two valve chambers 102b, 102c in the designed micropump 100, this pressure drop is double as known to those skilled in the art, and creates a required pressure for flow that is large compared to what can be expected in an ambulatory environment. This unanticipated pressure may come from, for example, changes in altitude or compression of the reservoir.

The use of hydraulic resistance solves the issue of free flow within the micropump 100, but consideration must be given that this resistance may require large amounts of pressure to move fluid through the valve gap when desired to pump, mainly during priming of the micropump. This is resolved by the active nature of the valve and the cubic relationship between hydraulic resistance and the valve gap height. During pumping, a valve is opened prior to the pump chamber actuating upwards. This changes the valve gap height to the unactuated gap height plus the amount of deflection or deformation achieved with the piezoelectric actuator, drastically reducing the pressure drop across the valve. And secondly, as the pump chamber drives downwards, the valve is able to close down on to the valve lips and actually make contact (or open at the outlet), and this is the flow rectifying portion of the active valve. This is required as the pressure generated in the main pump chamber is larger than that of what would be expected in an ambulatory environment that could generate free flow and therefore in the actuation sequence the valve is fully closed down on to the valve seat (lip) to prevent leakage.

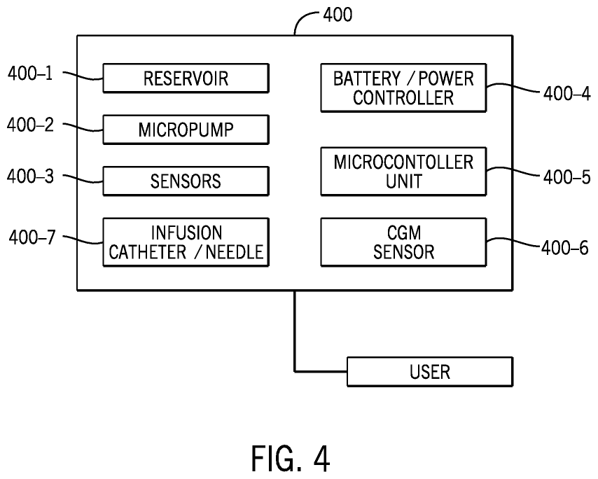

FIG. 4 depicts a block diagram of example components of device 400 for delivering insulin of an infusion system as described above. (Device 104 is renumbered as device 400 in FIG. 4.) Specifically, device 400 incorporates several components or modules (not shown) in the fluidic pathway including reservoir 400-1 for storing the insulin, micropump 400-2 (as described hereinabove) for pumping the insulin, sensors 400-3 (e.g., pressure) for sensing various parameters in the system and user and tubing connecting infusion catheter or infusion needle 400-7 to reservoir 400-1. Device 400 also includes microcontroller unit (MCU) 400-4 and battery and power controller 400-5.

Device 400 further includes CGM sensor 400-6. CGM or continuous glucose monitoring, as known to those skilled in the art, tracks user glucose levels and permits those levels to be used in algorithms that control flow rate. MCU 400-4 controls the operation of micropump 400-2. Infusion needle 400-7 and CGM sensor 400-6 are shown as separate components in FIG. 4 for illustration purposes. Infusion catheter or needle 400-7 and CGM sensor 400-6 may be integrated or may be separate (individually).

Reservoir 400-1 is configured to receive and store insulin for its delivery over a course of about three days, or as needed. However, reservoir size may be configured for storing any quantity of fluid as required.

MCU 400-5 electronically communicates with sensors 400-3 and micropump 400-2 as well as the CGM sensor 400-6, as the monitoring components. Among several functions, MCU 400-5 operates to control the operation of micropump 400-2 to deliver insulin through infusion catheter or infusion needle 400-7 from reservoir 400-1 at specific doses, i.e., flow rates over specified time intervals, based on CGM data converted to desired flow rate via control algorithms.

Battery and power controller 400-4 controls the power to MCU 400-5 and micropump 400-2 to enable those components to function properly as known to those skilled in the art. CGM sensor 400-2 is powered by battery and power controller 400-4 through MCU 400-5.

FIG. 4 depict device 400 with only a few components. Those skilled in the art know that device 400 include additional components.

It shall be understood that this disclosure teaches examples of the illustrative embodiments and that many variations of the invention can easily be devised by those skilled in the art after reading this disclosure and that the scope of the present invention is to be determined by the claims below.

What is claimed is:

1. A MEMS device for a device for delivering medicament to a user, the MEMS device configured as a valve for permitting or preventing a flow of the medicament to the user, the MEMS device comprising:
a first port and a second port to enable medicament to flow through the MEMS device;
first and second wafers that define a cavity therebetween that communicates with the first and second ports, thereby creating a fluid path for a flow of the medicament from the first port to the second port, the first wafer configured as a membrane adapted to deform, the cavity including a first chamber that communicates with the second port;
a first valve section including (1) a first valve seat around the first port that extends from the second wafer into the first chamber to a distal end thereof and (2) a valve gap that is defined as a distance between the distal end and the membrane, wherein the valve seat is configured so that a hydraulic resistance through the valve gap exceeds a hydraulic resistance in the valve section to ensure that medicament is prevented from flowing through the first port in the event the MEMS device has lost power; and
a first piezoelectric actuator layered on the first wafer and configured to cause the membrane to deform and close off the first valve seat, thereby preventing fluid flow through the first port.

2. The MEMS device of claim 1 wherein the valve seat has a width that is adjusted to increase the hydraulic resistance in the valve gap.

3. The MEMS device of claim 1 wherein the valve seat has a radius that is adjusted to increase the hydraulic resistance in the valve gap.

4. The MEMS device of claim 1 wherein the cavity includes a second chamber in communication with the first chamber and first port and the MEMS device further comprising a pump section including a second piezoelectric actuator that is layered on top of the first wafer and is configured to deform the first wafer into the second chamber to draw into or displace medicament into the cavity.

5. The device of claim 1 wherein the medicament is insulin.

6. A device for delivering medicament to a user including a MEMS device configured as a micropump for pumping the medicament into the user, the MEMS device comprising:
a first port and a second port to enable medicament to flow through the MEMS device;
first and second wafers that define a cavity therebetween that communicates with the first and second ports, thereby creating a fluid path for a flow of the medicament through the first port and the second port, the first wafer configured as a membrane that that is adapted to deform into the cavity, the cavity including a first chamber that communicates with the first port and a second chamber that communicates with the first chamber and the second port creating the fluid path and enabling the flow of medicament through the MEMS device;
a pump section including a first piezoelectric actuator that is layered on top of the first wafer and is configured to deform the first wafer into the first chamber to draw into or displace medicament into the first chamber; and
a first valve section including a first valve seat around the second port that extends from the second wafer into the second chamber to a distal end thereof and (2) a valve gap that is defined as a distance between the distal end and the membrane, wherein the valve seat is configured so that a hydraulic resistance through the valve gap exceeds a hydraulic resistance in the valve section to ensure that medicament is prevented from flowing through the second port in the event the MEMS device has lost power,
wherein the second valve section further includes a second piezoelectric actuator layered on the first wafer and configured to cause the membrane to deform and close off the first valve seat, thereby preventing fluid flow through the second port.

7. The MEMS device of claim 6 wherein the first valve seat has a width that is adjusted to increase the hydraulic resistance through the second valve gap.

8. The MEMS device of claim 6 wherein the first valve seat has a radius that is adjusted to increase the hydraulic resistance through the second valve gap.

9. The MEMS device of claim 6 wherein the cavity includes a third chamber in communication with the first chamber and first port and the MEMS device further comprising a second valve section including a second valve seat around the second port that extends from the second wafer into the third chamber to a distal end thereof and (2) a second valve gap that is defined as a distance between the distal end and the membrane, wherein the valve seat is configured so that a hydraulic resistance through the second valve gap exceeds a hydraulic resistance in the second valve section to ensure that medicament is prevented from flowing through the second port in the event the MEMS device has lost power.

10. The device of claim 6 wherein the second valve section further includes a second piezoelectric actuator layered on the first wafer and configured to cause the membrane to deform and close off the second valve seat, thereby preventing fluid flow through the second port.

11. The device of claim 6 wherein the medicament is insulin.

12. A method of actuating a MEMS device for a device for delivering medicament to a user, the MEMS device configured as a valve for permitting or preventing a flow of the medicament to the user, the MEMS device including a first port and a second port to enable medicament to flow through the MEMS device, first and second wafers that define a cavity therebetween that communicates with the first and second ports, thereby creating a fluid path for a flow of the medicament from the first port to the second port, the first wafer configured as a membrane, the cavity including a first chamber that communicates with the second port, the MEMS device further including (1) a first valve section with a first valve seat around the second port that extends from the second wafer into the second chamber to a distal end thereof, (2) a valve gap that is defined as a distance between the distal end and the membrane and (3) a first piezoelectric actuator layered on the first wafer to cause the membrane to deform and seal the second port, thereby preventing fluid flow through the second port, the method comprising:
adjusting the dimensions of the valve seat so that a hydraulic resistance through the valve gap exceeds a hydraulic resistance in the MEMS device to ensure that medicament is prevented from flowing through the second port in the event the MEMS device has lost power.

13. The method of claim 12 wherein adjusting includes adjusting a width of the valve seat to increase the hydraulic resistance through the valve gap.

14. The method of claim 12 wherein adjusting includes adjusting a radius of the valve seat to increase the hydraulic resistance through the valve gap.
